(19) United States
(12) Patent Application Publication
(10) Pub. No.: US 2016/0169901 A1
(43) Pub. Date: Jun. 16, 2016

(54) METHODS FOR DIAGNOSIS OF CUTANEOUS T-CELL LYMPHOMA

(71) Applicant: The Trustees of Columbia University in the City of New York, New York, NY (US)

(72) Inventors: Larisa J. GESKIN, Bronx, NY (US); Oleg E. AKILOV, Pittsburgh, PA (US)

(73) Assignee: The Trustees of Columbia University in the City of New York, New York, NY (US)

(21) Appl. No.: 14/965,594

(22) Filed: Dec. 10, 2015

Related U.S. Application Data
(60) Provisional application No. 62/090,166, filed on Dec. 10, 2014.

(51) Int. Cl. G01N 33/574

(52) U.S. Cl. CPC G01N 33/57426; G01N 2800/52; G01N 2333/70578; G01N 2333/5434

(57) ABSTRACT

The present invention is based, at least in part, on the discovery of biomarkers identified herein which are associated with cutaneous T-cell lymphoma (CTCL). Accordingly, the present invention provides methods for diagnosing CTCL, e.g., early stage CTCL, including mycosis fungoides (MF) and Sézary syndrome (SS), and to distinguish CTCL from other skin disorders.

### RELATED APPLICATIONS

This application claims priority to U.S. Provisional Patent Application No. 62/090,166, filed on Dec. 10, 2014, the entire contents of which is expressly incorporated herein by reference.

### GRANT INFORMATION

This invention was made with government support under Grant No. 5P50CA121973-03, awarded by the National Institutes of Health. The government has certain rights in the invention.

### BACKGROUND

T-cell lymphoma that involves the skin is generally known as cutaneous T-cell lymphoma (CTCL). The term CTCL encompasses a number of disorders, including mycosis fungoides (MF), an indolent lymphoma of the skin, which is the most common form of CTCL. Sézary syndrome (SS) is an advanced, variant form of mycosis fungoides, characterized by the presence of malignant lymphocytes in the blood (See “Getting the Facts” monograph for “Cutaneous T Cell Lymphoma” published by the Lymphoma Research Foundation, 115 Broadway Suite 1301, New York N.Y. 10006 (last update January 2013)). MF is a malignancy with annual overall incidence of 10.2 per million persons (Korgavkar K, Xiong M, Weinstock M. JAMA Dermatol. 2013; 149(11): 1295-1299).

CTCL is a slowly progressive disease that most often manifests by nonspecific erythematous eczematous patches and plaques of the skin. In early stages, it manifests by non-specific cutaneous patches and plaques, similar to benign dermatoses, such as eczema and psoriasis, earning it the name “the great imitator.” (Zackheim, et al. J. Am. Acad Dermatol 2002; 47(6):914-8). These nonspecific manifestations frequently lead to a misdiagnosis and/or a delay in the diagnosis of MF for many years, and result in more advanced stage at diagnosis and worse clinical outcomes. (Arai E, Katayama I, Ishihara K. Clinicopathologic study of 107 autopsy cases. Pathol Res Pract. 1991; 187(4):451-457). Prognosis and survival of CTCL patients are influenced greatly by the stage at diagnosis (Kim Y H, Hoppe R T. Semin Oncol. 1999; 26(3):276-289; Foss F M, Sausville E A. Hematol Oncol Clin North Am. 1995; 9(5):1011-1019). Accordingly, survival of patients with minimal patch stage skin involvement is similar to age-matched control patients, whereas patients with cutaneous tumors have a 3 year median survival (Duvic M, Apisarnthanarax N, Cohen D S, Smith T L, Ha C S, Kurzrock R. J Am Acad Dermatol. 2003; 49(1):35-49). Therefore, early detection can improve clinical outcomes.

No specific diagnostic or prognostic markers exist to enable early diagnosis of MF, SS and CTCL. Diagnosis of MF is difficult due to lack of specific clinical, histological or molecular markers for this malignancy (Nashan, D., Faulhaber, D., Stander, S., Luger, T. A. and Stadler, R. (2007), British Journal of Dermatology, 156: 1-10. doi: 10.1111/j.1365-2133.2006.07526.x). The skin biopsy in many cases is unreliable since, histologically, early CTCL mimics benign inflammatory dermatoses, such as psoriasis or eczema (Santucci M, Biggeri A, Feller A C, Burg G. Arch Dermatol. 2000; 136(4):497-502; Zemheri E, Ozkanli S, Zindanci I, et al. Scientific World Journal. 2012; 2012:426732). Furthermore, the biopsy interpretation of CTCL is frequently highly subjective, and published studies have demonstrated poor reproducibility of diagnoses rendered by different pathologists in evaluating biopsy specimens of early CTCL (Lefeber W P, Robinson J K, Clendenning W E, Dunn J L, Colton T. Arch Dermatol. 1981; 117(7):408-411; Olerud J E, Kulin P A, Chew D E, et al. Arch Dermatol. 1992; 128(4):501-507).

Molecular biology techniques may be helpful in the differential diagnosis. However, clonal rearrangements of the T-cell receptor-γ gene by polymerase chain reaction is positive only in 74% of those biopsy samples diagnostic of early MF, and requires numerous invasive skin biopsies. (Tok J, Szabolcs M J, Silvers D N, Zhong J, Matsushima A Y. J Am Acad Dermatol. 1998; 38(3):453-460). Considering the low prevalence of MF, a screening strategy must achieve high specificity and sensitivity to avoid an unacceptable level of false-positive results.

Detection of early disease and appropriate therapeutic intervention at the early stage would prevent poor clinical outcomes and avert the use of potentially life-threatening medications in MF patients. If diagnosed early, CTCL patients could be treated appropriately. Thus, a diagnostic test that can easily confirm the presence of CTCL early in disease course is needed.

### SUMMARY

The present invention is based, at least in part, on the discovery of biomarkers identified herein which are associated with cutaneous T-cell lymphoma (CTCL), including early stage CTCL. Accordingly, the present invention provides methods for diagnosing CTCL, e.g., early stage CTCL, including mycosis fungoides (MF) and Sézary syndrome (SS), and to distinguish CTCL from other skin disorders.

In one aspect, the present invention is directed to methods of determining whether a subject has CTCL, the method comprising determining the level of one or more biomarkers as described herein in a biological sample, e.g., a plasma or serum sample, obtained from the subject relative to the level of expression in a control sample, wherein increased expression of the one or more of the biomarkers indicates a diagnosis of CTCL in the subject. In one embodiment, the method further comprises treating the subject for CTCL.

In another aspect, the present invention is directed to methods of determining whether a subject has CTCL, the method comprising determining the levels of three or more biomarkers in a biological sample, e.g., a plasma or serum sample, obtained from the subject relative to the level of expression of the three or more biomarkers in a control sample, wherein the biomarkers comprise TNFR1, TNFR2, and IL12p40/70, and wherein increased expression of the three or more biomarkers indicates a diagnosis of cutaneous T-cell lymphoma in the subject. In one embodiment, the method further comprises treating the subject for CTCL.

In another aspect, the present invention is directed to methods of excluding a diagnosis of CTCL in a subject, the method comprising determining the level of one or more biomarkers as described herein in a biological sample, e.g., a plasma or serum sample, obtained from the subject relative to the level of expression in a control sample, wherein normal expression of the one or more biomarkers excludes a diagnosis of CTCL in the subject. In one embodiment, the method further comprises treating the subject with a therapeutic agent that is useful for the treatment of a skin disorder other than CTCL.

In another aspect, the present invention is directed to methods of excluding a diagnosis of cutaneous T-cell lymphoma (CTCL), or an associated disorder, in a subject, comprising determining the levels of three or more biomarkers in a biological sample, e.g., a plasma or serum sample, obtained from the subject relative to the level of expression of the three or more biomarkers in a control sample, wherein the biomarkers comprise TNFR1, TNFR2, and IL12p40/70, and wherein normal levels of the three or more biomarkers excludes a diagnosis of CTCL. In one embodiment, the subject is treated with a therapeutic agent that is useful for the treatment of a skin disorder other than CTCL.

In yet another aspect, the present invention provides methods of assessing the efficacy of a therapy for treating CTCL in a subject, comprising determining the level of one or more biomarkers as described herein in a biological sample obtained, e.g., a plasma or serum sample, from the subject, prior to therapy with a therapeutic agent; and determining the level of the one or more biomarkers in a biological sample obtained from the subject, at one or more time points during therapy with the therapeutic agent, wherein the therapy with the therapeutic agent is efficacious for treating the CTCL in the subject when there is a lower level of the one or more biomarkers in the second or subsequent samples, relative to the first sample.

In yet another aspect, the present invention is directed to methods of assessing the efficacy of a therapy for treating cutaneous T-cell lymphoma (CTCL) in a subject, comprising determining the levels of three or more biomarkers in a biological sample, e.g., a plasma or serum sample, obtained from the subject, prior to therapy with a therapeutic agent, wherein the biomarkers comprise TNFR1, TNFR2, and IL12p40/70; and determining the levels of the three or more biomarkers in a biological sample obtained from the subject, at one or more time points during therapy with the therapeutic agent, wherein the therapy with the therapeutic agent is efficacious for treating the cutaneous T-cell lymphoma in the subject when there is a lower level of the three or more biomarkers in the second or subsequent samples, relative to the first sample.

In some embodiments, the foregoing aspects of the invention further comprise the step of performing an additional CTCL detection method.

In one embodiment of the foregoing aspects of the invention, the subject is a human. In another embodiment of the foregoing aspects of the invention, the subject has a skin lesion, e.g., a patch or plaque. In another embodiment of the foregoing aspects of the invention, the cutaneous T-cell lymphoma (CTCL) is mycosis fungoides (MF) or Sézary syndrome (SS). In another embodiment of the foregoing aspects of the invention, the CTCL is early stage CTCL, e.g., early stage MF or SS.

In certain embodiments, the biomarkers used in the methods of the invention include one or more (or two or more, or three or more, or four or more, or five or more, or six or more, or seven or more, or eight) of CXCL9, CXCL10, IL-12p40/70, CCL11, sIL-2R, CCL2, TNFR1, and TNFR2. In another embodiment, the biomarkers include a panel of at least three of CXCL9, CXCL10, IL-12p40/70, CCL11, sIL-2R, CCL2, TNFR1, and TNFR2. In another embodiment, the panel of biomarkers comprises TNFR1, TNFR2, and IL-12p40/70.

In other non-limiting embodiments, the biomarkers include a panel of at least four of CXCL9, CXCL10, IL-12p40/70, CCL11, sIL-2R, CCL2, TNFR1, and TNFR2.

In another non-limiting embodiment, biomarkers used in the invention include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In another non-limiting embodiment, the biomarkers include a panel of three or four biomarkers selected from the panels of biomarkers identified in Table 4.

In certain non-limiting embodiments, the biological sample can be a blood sample. In a related embodiment, the biological sample is a plasma or a serum sample. In specific non-limiting embodiments, one or more biomarkers can be detected in one or more biological samples from a subject.

In one embodiment, the biomarker is a protein and the presence of the protein is detected by contacting a sample with a reagent which specifically binds with the protein. For example, the reagent can be selected from the group consisting of an antibody, an antibody derivative, an antigen-binding antibody fragment and a non-antibody peptide which specifically binds the protein. In another embodiment, the antibody or antigen-binding antibody fragment is a monoclonal antibody or antigen-binding fragment thereof, or a polyclonal antibody or antigen-binding fragment thereof. In one embodiment, contacting the sample with the reagent transforms the sample in a manner such that the level of expression of the biomarker(s) is detected and quantified.

In another embodiment, the biomarker can also be a transcribed polynucleotide or portion thereof, e.g., a mRNA, and the presence of the polynucleotide is detected by contacting a sample with one or more probes, primers or other detection reagents for detecting one or more biomarkers of the present invention. In one embodiment, detecting a transcribed polynucleotide includes amplifying the transcribed polynucleotide. In another non-limiting embodiment, the nucleic acid biomarker can be detected by RNA in situ hybridization. In one embodiment, contacting the sample with the reagent transforms the sample in a manner such that the level of expression of the biomarker(s) is detected and quantified.

The invention also provides kits for diagnosing or assessing whether or not a subject has CTCL, e.g., early CTCL, for monitoring the therapeutic treatment of a subject, or for assessing the efficacy of a therapeutic treatment regime of a subject, where the kit containing reagents useful for detecting the level of expression of biomarkers in a biological sample, e.g., a blood sample, e.g., a plasma or serum sample.

In one embodiment of the foregoing aspects of the invention, the subject is a human. In another embodiment of the foregoing aspects of the invention, the subject has a skin lesion, e.g., a patch or plaque.

In another embodiment of the foregoing aspects of the invention, the cutaneous T-cell lymphoma (CTCL) is mycosis fungoides (MF) or Sézary syndrome (SS). In another embodiment of the foregoing aspects of the invention, the CTCL is early stage CTCL, e.g., early stage MF or SS.

In certain embodiments, the biomarkers used in the kits of the invention include one or more (or two or more, or three or more, or four or more, or five or more, or six or more, or seven or more, or eight) of CXCL9, CXCL10, IL-12p40/70, CCL11, sIL-2R, CCL2, TNFR1, and TNFR2. In another embodiment, the biomarkers used in the kits of the invention include a panel of at least three of CXCL9, CXCL10, IL-12p40/70, CCL11, sIL-2R, CCL2, TNFR1, and TNFR2. In another embodiment, the panel of biomarkers comprises TNFR1, TNFR2, and IL-12p40/70.

In other non-limiting embodiments, the biomarkers used in the kits of the invention include a panel of at least four of CXCL9, CXCL10, IL-12p40/70, CCL11, sIL-2R, CCL2, TNFR1, and TNFR2.

In another non-limiting embodiment, biomarkers used in the kits of the invention include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In another non-limiting embodiment, the biomarkers used in the kits of the invention include a panel of three or four biomarkers selected from the panels of biomarkers identified in Table 4.

### BRIEF DESCRIPTION OF THE FIGURES

FIG. 1 is a flowchart of experimental design as exemplified by Example 1 as set forth herein.

FIGS. 2A-2B. FIG. 2A depicts the level of IL-12p40/p70, TNFR1, and TNFR2 in the peripheral blood of patients with MF, psoriasis or healthy controls. (*, p<0.05, ***, p<0.001). FIG. 2B depicts the age-dependent level of IL-12p40/p70, TNFR1, and TNFR2 in the peripheral blood of patients with MF and age-matched controls.

FIG. 3 depicts cumulative receiver operating characteristic (ROC) curves in the validation sets using the three-biomarker panel versus IL-12p40/70 biomarker only in healthy controls versus patients with MF.

FIGS. 4A-4B depict the overall survival (OS) of patients with MF in relation to levels of Il-12p40/70 (FIG. 4A) or TNFR1 (FIG. 4B). The survival curves (Kaplan-Meier plots) show a comparison of the OS of patients with MF with IL-12p40/70 (FIG. 4A) or TNFR1 (FIG. 4B) serum levels. According to the respective median, patients with serum levels of IL-12p40/70 >280 pg/ml are considered as high producers and patients with <280 pg/ml serum as low producers. Patients with serum levels of TNFR1 >2000 pg/ml are considered as high producers and patients with <2000 pg/ml serum as low producers. High serum levels of both biomarkers are associated with shorter survival periods. P refers to the log-rank (Mantle-Cox) test.

FIGS. 5A-5B depict IL12p70 in biopsy samples from patients with patch MF and psoriasis. FIG. 5A. IL-12p70 staining was performed as outlined in Example 1. ×20 and ×100 (inserts). (Representative figures). Note extracellular localization of IL-12p70 characteristic of cytokine pattern. FIG. 5B. IHC score: 5 patients with patch MF and 5 patients with psoriasis. *, p<0.05.

FIGS. 6A-6D. FIG. 6A illustrates that malignant lymphocytes are characterized by profound loss of CD26 expression. Cells of peripheral blood were gated on lymphocyte fraction in FSS/SCC. Isolated fractions of lymphocytes with magnetic beads were stained with Diff-Quik. CD4+CD26− cells derived from patients with CTCL revealed convoluted cerebriform nuclei characteristic of malignant cells. Light microscopy, ×1000. FIG. 6B shows representative flow of TNFR1 and TNFR2 expression on CD4⁺CD26⁺ and CD4⁺CD26⁻ lymphocytes from peripheral blood of patient with SS. FIG. 6C illustrates the percentage of TNFR1 or TNFR2 expression on lymphocytes from 15 patients with SS (*, p<0.0001 in comparison with CD4⁺CD26⁺). FIG. 6D. Surface staining for TNFR1 on lymphocytes in psoriasis patients is absent in Pauteie microabscesses in patients with patch MF. (×100).

### DETAILED DESCRIPTION

Differential diagnosis of early stage of CTCL is a clinically challenging task since many patients present with non-specific eczematous patches which mimic many benign disorders, such as eczema, psoriasis, contact dermatitis and other benign dermatoses, and, thus, misdiagnosis is common. As described herein, the present invention provides methods for diagnosing CTCL, e.g., early stage CTCL, including mycosis fungoides (MF) and Sézary syndrome (SS). In particular, the present invention relates to the identification and use of biomarkers identified herein having a high level of sensitivity and specificity to diagnose CTCL in a subject, to exclude a diagnosis of CTCL in a subject, and/or to distinguish CTCL from other skin disorders, using a biological sample, e.g., a blood sample (including a serum or plasma sample), obtained from the subject.

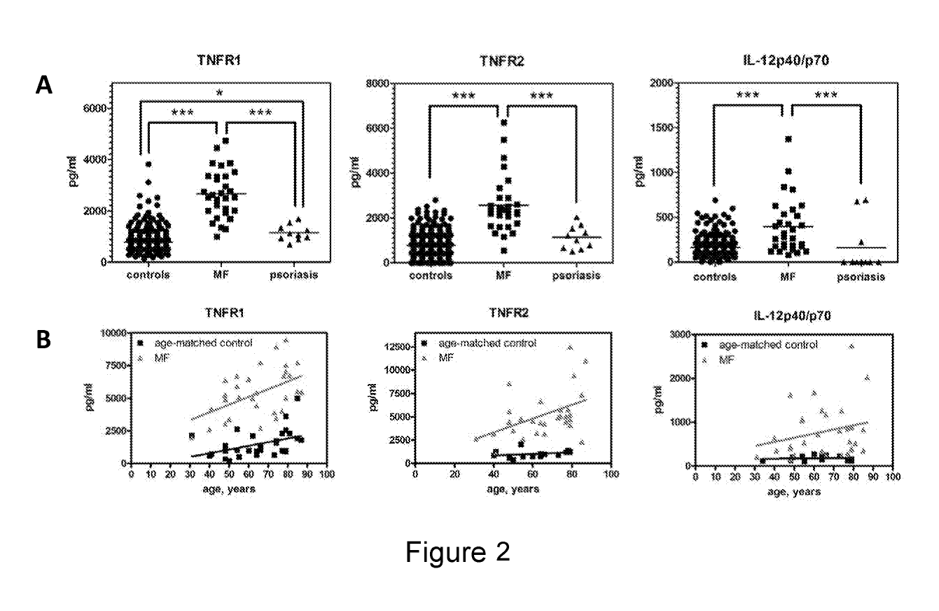

As described in detail in Example 1, below, thirty-four serum biomarkers were analyzed in sera from 30 patients with MF, 10 patients with psoriasis, and 756 healthy volunteers. To rule out age as a confounder, age-matched controlled analysis was performed. Controlling for age, expression levels differ significantly among cases and controls for the following 8 biomarkers: CCL11, IL-12p40/p70, sIL-2R, IP10, CCL2, TNFR1, TNFR2, and CXCL9 (p<0.05 for each). The difference in level of IL-12p40/70, CCL2, TNFR1, and TNFR2 between MF patients with early disease and age-matched controls was distinct (p<0.001). To identify a biomarker panel discriminating early-stage MF from healthy controls, cross validation was performed against 90 randomly selected healthy controls. Ten patients with psoriasis comprised another group of validation (see FIG. 2). A three-biomarker panel providing the highest diagnostic power of 85% sensitivity for all stages of MF and 72% sensitivity for early-stage MF at 98% specificity was found which includes the biomarkers TNFR1, TNFR2, and IL12p40/70. While these three biomarkers provide high sensitivity and specificity, the present invention is not limited to the use of this three biomarker panel, as further described herein.

Thus, in certain non-limiting embodiments, the present invention provides methods of diagnosing CTCL, including early CTCL, or an associated disorder, in a subject, comprising determining the level of expression of one or more biomarkers, e.g., the biomarkers TNFR1, TNFR2, and IL12p40/70, in a sample of a subject, relative to a normal healthy subject, where increased expression in one or more, or all three, of these biomarkers indicates a diagnosis of CTCL. In one embodiment, the subject tested displays nonspecific cutaneous patches or plaques. In another embodiment, one or more additional CTCL detection methods, such as skin biopsy or molecular testing, can be carried out. In still another embodiment, a CTCL therapeutic agent is administered to the subject to thereby treat CTCL, e.g., MF or SS, in the subject.

An accurate diagnosis either confirming or excluding CTCL is important in ensuring overall prognosis and disease monitoring recognizing disease progression early (such as lymph node, blood, or visceral organ involvement by lymphoma) and intervening appropriately. Moreover, where a subject has CTCL, e.g., MF or SS, and is misdiagnosed as having a benign dermatoses, for example, psoriasis or eczema, drugs contraindicated for CTCL may be administered, thus leading to CTCL disease propagation and potentially death (Corazza, M., Zampino, et al. Acta Derm Venereol 90, 616-620 (2010); Jacks, S. M. et al. J Am Acad Dermatol 71, e86-87 (2014); and Quereux, G. et al. Acta Derm Venereol 90, 616-620 (2010)).

Drugs that are routinely used in the treatment of benign skin disorders, e.g., psoriasis or eczema, but are detrimental for use in subjects with CTCL include, but are not limited to, tumor necrosis factor-α (TNF-α) antagonists, such as HUMIRA® (adalimumab), Enbrel® (etanercept), Remicade® (infliximab), Simponi® (golimumab), Cimzia® (certolizumab pegol), or other therapeutics such as Stelara® (ustekinumab), Cosentyx® (secukinumab), as well as calcineurin inhibitors, including cyclosporin, pimecrolimus and tacrolimus, or combinations thereof.

Drugs that are in development for the treatment of psoriasis but are potentially detrimental for use in subjects with CTCL include, but are not limited to, Guselkumab (CNTO 1959) (Janssen), HUMIRA® biosimilar candidates including ABP 501 (Amgen) and GP 2017 (Sandoz Pharmaceuticals), Tregalizumab (BT-061) (Biotest/AbbVie), Tildrakizumab (MK-3222/SCH 900222) (Merck), Namilumab (Takeda), IMO-8400 (Idera Pharmaceuticals), BI 655066 (Boehringer-Ingelheim), Brodalumab (Valeant Pharmaceuticals and AstraZeneca), XP23829 (XenoPort), KD025 (Kadmon Corporation), Alitretinoin (Stiefel), ASP015K (Janssen), Apo805K1 (ApoPharma), FP187 (Forward-Pharma), LEO 22811 (Leo Pharama), JAK inhibitors, such as Tofacitinib (Xeljanz or Jakvinus) (Pfizer) and Baricitinib (Eli Lilly/Incyte), VB-201 (VBL Therapeutics), Otezla (apremilast) (Celgene).

Thus, the diagnostic methods of the present invention are useful in preventing the adverse effects associated with treatment using one or more of these therapeutic agents when the subject is suffering from CTCL, but has been misdiagnosed as having a benign skin disorder such as psoriasis.

The present invention also provides methods of excluding a diagnosis of CTCL, or an associated disorder, in a subject, comprising determining the level of expression of one or more biomarkers, e.g., the biomarkers TNFR1, TNFR2, and IL12p40/70, in a sample of a subject, relative to a normal healthy subject, where normal levels of one or more of these biomarkers excludes a diagnosis of CTCL. Subsequently, the subject can be treated with a therapeutic agent that is useful for the treatment of a skin disorder but contraindicated in CTCL, e.g., a TNF-α-inhibitor or a calcineurin inhibitor.

In some embodiments, the present invention provides methods for screening potential participants in clinical trials. In particular, in one embodiment, the present invention provides methods for identifying subjects who either have or do not have CTCL prior to acceptance into a clinical trial or monitoring subject during the course of a clinical trial. For example, in one embodiment, in a clinical trial for a therapeutic agent to treat a benign skin disease, such as psoriasis, the methods of the invention can be used to exclude potential subjects from the trial based on increased expression of the biomarkers of the invention which indicates a diagnosis of CTCL. In another embodiment, in a clinical trial for a therapeutic agent to treat CTCL, the methods of the invention can be used to confirm that potential subjects have CTCL based on increased expression of the biomarkers of the invention.

In certain, non-limiting embodiments, the biomarkers used in the methods of the invention include one or more (or two or more, or three or more, or four or more, or five or more, or six or more, or seven or more, or eight) of the biomarkers comprising CXCL9, CXCL10, IL-12p40/70, CCL11, sIL-2R, CCL2, TNFR1, and TNFR2. In another non-limiting embodiment, the biomarkers used in the methods of the invention include a panel of at least three of CXCL9, CXCL10, IL-12p40/70, CCL11, sIL-2R, CCL2, TNFR1, and TNFR2. In another non-limiting embodiment, the panel of biomarkers comprises TNFR1, TNFR2, and IL-12p40/70.

In other non-limiting embodiments, the biomarkers tested include a panel of at least four of CXCL9, CXCL10, IL-12p40/70, CCL11, sIL-2R, CCL2, TNFR1, and TNFR2.

In another non-limiting embodiment, the biomarkers tested include a panel of three or four biomarkers selected from the panels of biomarkers identified in Table 4 (set forth in Example 1, below).

In another non-limiting embodiment, biomarkers used in the invention to identify early CTCL, e.g., early MF or SS, include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In one embodiment, when a panel is used, a diagnosis of CTCL in the subject is based on an increase in the expression levels of each of the biomarkers in the panel. In another embodiment, when a panel is used, a diagnosis of CTCL in the subject is based on an increase in the expression levels of one or more of the biomarkers in the panel.

In some specific, non-limiting embodiments, the following biomarkers may have the exemplary UniProt Accession Nos. identified in Table 1, below.

TABLE 1

| | UniProt |
|---|---|
| Biomarker | Accession No. |
| C-X-C motif chemokine 9 | Q07325 |
| (CXCL9) | |
| C-X-C motif chemokine 10 | P02778 |
| (CXCL10) | |
| Interleukin-12 subunit beta (IL12B) | P29460 |
| (p40)/(IL-12p40/70) | |
| Eotaxin (CCL11) | P51671 |
| sIL-2R: | |
| Interleukin-2 receptor subunit alpha | P01589 |
| (IL2RA) | |
| Interleukin-2 receptor subunit beta | P14784 |
| (IL2RB) | |
| C-C motif chemokine 2 (CCL2) | P13500 |
| Tumor necrosis factor receptor 1 | P19438 |
| (TNFR1) | |
| Tumor necrosis factor receptor 2 | P20333 |
| (TNFR2) | |

C-X-C motif chemokine 9 (CXCL9) is also referred to as gamma-interferon-induced monokine, monokine induced by interferon-gamma, HuMIG, MIG, and small-inducible cytokine B9.

C-X-C motif chemokine 10 (CXCL10) is also referred to as 10 kDa interferon gamma-induced protein, gamma-IP10, IP-10, and small-inducible cytokine B10.

Interleukin-12 subunit beta (IL12B) (p40)/(IL-12p40/70), is also referred to as cytotoxic lymphocyte maturation factor 40 kDa subunit, CLMF p40, IL-12 subunit p40, NK cell stimulatory factor chain 2, and NKSF2.

Eotaxin (CCL11) is also referred to as C-C motif chemokine 11, eosinophil chemotactic protein, and small-inducible cytokine A11.

Soluble human interleukin-2 receptor (sIL-2R; CD25), is comprised of at least two subunits, referred to as Interleukin-2 receptor subunit alpha (IL2RA) and Interleukin-2 receptor subunit beta (IL2RB). In one embodiment, the alpha subunit of sIL-2R is used as a biomarker in the methods of the invention. In another embodiment, the beta subunit of sIL-2R is used as a biomarker in the methods of the invention.

C-C motif chemokine 2 (CCL2) is also referred to as HC11, monocyte chemoattractant protein 1, monocyte chemotactic and activating factor, MCAF, monocyte chemotactic protein 1, MCP-1, monocyte secretory protein JE, and small-inducible cytokine A2.

Tumor necrosis factor receptor 1 (TNFR1) is also referred to as tumor necrosis factor receptor superfamily member 1A (TNFRSF1A), Tumor necrosis factor receptor type I, TNF-RI, p55, p60, CD_antigen and CD120a. TNFR1 is cleaved into the following 2 chains: Tumor necrosis factor receptor superfamily member 1A, membrane form and Tumor necrosis factor-binding protein 1.

Tumor necrosis factor receptor 2 (TNFR2) is also referred to as tumor necrosis factor receptor superfamily member 1B (TNFRSF1B), Tumor necrosis factor receptor type II, TNF-RII, TNFR-II, p75, p80 TNF-alpha receptor, CD120b. TNFR2 is cleaved into the following 2 chains: Tumor necrosis factor receptor superfamily member 1b, membrane form and Tumor necrosis factor-binding protein 2 (TBP-2) and (TBPII).

Protein and nucleic acid variants, cleavage forms, and alternate isoforms of the biomarkers disclosed herein can be used in the methods of the invention.

In related embodiments, the invention provides for kits comprising a means, e.g., a reagent, capable of determining expression levels of the biomarkers of the invention, optionally together with a positive control. Such means may comprise, for example but not by way of limitation, an antibody or fragment thereof or single chain antibody specific for the biomarker or biomarkers to be detected; these may be directly detectable themselves or indirectly detectable, for example using a labeled secondary antibody or probe or a substrate. Other means, e.g., a reagent, capable of determining expression levels of the biomarkers of the invention include, but are not limited to, bead-based multiplexing technology, e.g., xMAP® technology (Luninex Corporation), packaged probe and primer sets (e.g., TaqMan probe/primer sets), arrays/microarrays, biomarker-specific antibodies and beads, which further contain one or more probes, primers or other detection reagents for detecting one or more biomarkers of the present invention.

Furthermore, the effectiveness of CTCL therapy can be monitored by evaluating the presence and levels of the one or more of the biomarkers of the invention over the course of therapy, and decisions can be made regarding the type, duration, and course of therapy based on these evaluations.

### DEFINITIONS

As used herein, the term “biomarker” refers to both a marker (e.g., an expressed gene, including mRNA and/or protein) or a panel of markers, that allows detection of a disease in an individual, including detection of disease in its early stages. In one embodiment, biomarkers, as used herein, include nucleic acid and/or proteins, selected from the biomarkers comprising CXCL9, CXCL10, IL-12p40/70, CCL11, sIL-2R, CCL2, TNFR1, and TNFR2. In certain, non-limiting embodiments, a biomarker includes a panel of at least three of the biomarkers comprising CXCL9, CXCL10, IL-12p40/70, CCL11, sIL-2R, CCL2, TNFR1, and TNFR2. In another embodiment, the panel of biomarkers includes at least TNFR1, TNFR2, and IL-12p40/70.

In other non-limiting embodiments, the biomarkers include a panel of at least four of the biomarkers comprising CXCL9, CXCL10, IL-12p40/70, CCL11, sIL-2R, CCL2, TNFR1, and TNFR2.

In another non-limiting embodiment, the biomarkers include a panel of three or four biomarkers selected from the panels of biomarkers identified in Table 4.

In another, non-limiting embodiment, biomarkers used in the invention include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In specific embodiments, the expression level of biomarkers as determined by protein or nucleic acid levels in biological sample from an individual to be tested is compared with respective levels in normal biological sample from a control, e.g., a healthy individual. In certain non-limiting embodiments, a biomarker is a released and/or secreted protein that can be detected in a biological sample of a subject. For example, a biomarker can be shed from a malignant cell.

As used herein, the term “control” refers to any entity used in comparison of biomarker expression. For example, in one embodiment, a control can be the expression pattern of the biomarkers in an individual not affected by the disease. In another embodiment, a control can be the averaged expression pattern of the biomarkers from a group or population of individuals not affected by the disease. In another embodiment, a control can be the expression of another gene/protein in the same individual. In another embodiment, a control can be a threshold on the score produced by a mathematical model that uses the expressions of biomarkers and possibly expression of other genes/proteins so that scores for disease-affected individuals and for individuals not affected by the disease significantly differ. The expression and the expression pattern can be either absolute or relative, i.e., determined relative to the expression of some other gene(s)/protein(s). In specific embodiments, the control is derived at least in part from the level of expression of one or more reference genes or proteins from a single individual without CTCL. In another embodiment, the control is derived at least in part from the level of expression of one or more reference genes or proteins from a population of individuals without CTCL, e.g., the average level of expression. One of skill in the art recognizes that the control expression level may be normalized by standard means in the art. The normalization may include standardization to a reference protein (such as a housekeeping gene including GAPDH), for example (see also Tunbridge et al., 2011; Bar et al., 2009). In certain embodiments, the identification of CTCL, e.g., MF or SS, is achieved when the level of expression of a biomarker is above a normalized threshold compared to a control.

As used herein, the term “biological sample” refers to a sample of biological material obtained from a subject, preferably a human subject, including a biological fluid, e.g., blood (including serum or plasma).

The term “patient” or “subject,” as used interchangeably herein, refers to any warm-blooded animal, preferably a human. In non-limiting embodiments, the subject has a skin lesion, e.g., a patch or plaque. In other non-limiting embodiments, the subject has blood and/or bone marrow involvement. In non-limiting embodiments, the subject has extracutaneous involvement (lymph nodes and visceral organ metastases). In other non-limiting embodiments, the subject has a skin lesion and extracutaneous involvement.

The term “CTCL” as described herein refers to a number of disorders, including mycosis fungoides (MF), which is the most common form of CTCL and Sézary syndrome (SS), which is another variant form of CTCL, characterized by the presence of malignant lymphocytes in the blood, lymph node involvement by lymphoma, and/or diffuse skin involvement. (See, “Getting the Facts” monograph for “Cutaneous T Cell Lymphoma” published by the Lymphoma Research Foundation, 115 Broadway Suite 1301, New York N.Y. 10006 (last update January 2013)). CTCL is an extranodal, indolent non-Hodgkin lymphoma of T cell origin that primarily develops in the skin, but can involve the lymph nodes, blood, and visceral organs. In one embodiment, the term “CTCL” can be used interchangeably to refer to either MF or SS.

The clinical presentation of MF is highly variable. Cutaneous manifestations of the disease result from skin infiltration of malignant lymphocytes and depend on the extent of skin involvement. MF may progress through distinct stages of skin involvement, ranging from patch to plaque to tumor, but it may never progress or lesions may arise de novo. For descriptive purposes, the skin manifestations of early MF are divided into patch stage (patch-only disease) or plaque stage (both patches and plaques). A patch can be a flat lesion with various degrees of erythema and fine scaling; it may be atrophic or poikilodermatous, containing areas of hyperpigmentation, hypopigmentation, atrophy, and/or telangiectasias. A plaque is a demarcated erythematous, brownish, or violaceous lesion with a variable amount of scale. Distribution of the lesions depends on the clinical stage at presentation.

In earlier stages, the lesions have a predilection for folds and non-sun-exposed body areas (“bathing trunk” distribution). Progression through the stages is variable but commonly occurs over several years (see Epstein E H, Jr., Levin D L, Croft J D, Jr., Lutzner M A. Medicine (Baltimore) 1972; 51:61-72). Lesions usually are associated with pruritus, which may range from mild to excruciatingly severe, leading to insomnia, weight loss, depression, and suicidal ideations. Erythrodermic skin involvement occurs in 5 percent of patients with MF. Manifestations range from very faint to severe, with significant scaling, keratoderma, painful fissures of the hands and feet, nail dystrophy, and nail loss leading to the patient's inability to walk and maintain daily activities. Severely inflamed skin is a breeding ground for bacteria and other pathogens, with resulting fevers, chills, and septicemia. Extremity peripheral edema may be significant in the later stages and lead to cardiovascular compromise. The symptomotology and presentation usually reflects the site and severity of involvement and ranges from completely asymptomatic to severe pain, organ malfunction, or at the end stage disease multi-organ failure.

The term “early CTCL” or “early stage CTCL” refers to CTCL, e.g., MF or SS, at stages 1A to IIA, or where cutaneous papules, patches and/or plaques are visible with limited, if any, lymph node involvement and no visceral involvement. In one embodiment, in early CTCL, patients initially may present with “chronic dermatitis” that is resistant to therapy, which may be misdiagnosed as spongiotic dermatitis (so-called eczema), “psoriasis-like dermatitis,” or other chronic, nonspecific dermatoses, which may be associated with pruritus (itching). In the early stages of the disease, abnormal atypical infiltrate can be minimal and can be masked by normal inflammatory infiltrates in the skin or it can be misinterpreted as normal inflammatory infiltrate because of its mature CD4⁺ phenotype. Therefore, diagnosis may be difficult.

### Prognostic and Diagnostic Methods

Embodiments of the present invention relate to methods for diagnosing CTCL in a subject, including early stage CTCL, e.g., MF or SS. In one particular embodiment, a method for diagnosing CTCL in a subject is disclosed, wherein the method includes: (a) obtaining a biological sample from the subject; (b) determining a difference (e.g., an increase) in the level of expression of one or more biomarkers in the biological sample as compared to a control or reference sample; and (c) diagnosing CTCL in the subject, wherein a difference, e.g., increase, in the level of expression of the one or more biomarkers correlates to a positive diagnosis of CTCL in the subject.

The biomarkers that can be used in the methods of the present invention include one or more markers selected from CXCL9, CXCL10, IL-12p40/70, CCL11, sIL-2R, CCL2, TNFR1, and TNFR2. In certain, non-limiting embodiments, biomarkers include a panel of at least three of CXCL9, CXCL10, IL-12p40/70, CCL11, sIL-2R, CCL2, TNFR1, and TNFR2. In another embodiment, the panel of biomarkers includes at least TNFR1, TNFR2, and IL-12p40/70.

In another, non-limiting embodiment, biomarkers used in the invention include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In other non-limiting embodiments, the biomarkers include a panel of at least four of CXCL9, CXCL10, IL-12, CCL11, sIL-2R, CCL2, TNFR1, and TNFR2.

In another non-limiting embodiment, the biomarker includes a panel of three or four biomarkers selected from the panels of biomarkers identified in Table 4.

In addition, although previous studies (see, e.g., Wasik, Mariusz A. et al. Arch Dermatol. 1996; 132(1):42-47) have suggested that sIL-2R should not be used for diagnosis of early stage of CTCL, the inventors have surprisingly discovered that the increase in sIL-2R, together with other positive markers, is indicative of early stage CTCL. Thus, in another non-limiting embodiment, biomarkers used in the invention to identify early CTCL, e.g., early MF or SS, include three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In one embodiment, where a panel of biomarkers is used, expression of all of the biomarkers in the panel must be increased as compared to a control in order for the diagnosis of CTCL in the subject to be made.

In one embodiment, a method for diagnosing CTCL in the subject includes obtaining at least one biological sample from the subject. In various embodiments the one or more biomarkers, e.g., TNFR1, TNFR2, and IL-12p40/70, can be detected in blood (including plasma or serum). The step of collecting a biological sample can be carried out either directly or indirectly by any suitable technique. For example, a blood sample from a subject can be carried out by phlebotomy or any other suitable technique, with the blood sample processed further to provide a serum sample or other suitable blood fraction.

In another embodiment of the present invention, the method of diagnosing or screening for CTCL in a subject comprises, (a) obtaining a biological sample from the subject; (b) determining the presence of one or more biomarkers of the present invention, e.g., TNFR1, TNFR2, and IL-12p40/70, in a biological sample of the subject, wherein a difference (e.g., an increase) in the level of expression of one or more biomarkers as compared to a control or reference sample, indicates the presence of CTCL in the subject.

In some embodiments, the control or reference sample can be obtained, for example, from a normal biological sample of the subject or from a non-diseased, healthy subject.

In certain embodiments of the present invention, the method of diagnosing a subject with CTCL comprises determining a difference in the level of expression of biomarkers in a panel of biomarkers in a biological sample from the subject, as compared to a control, wherein the panel of biomarkers is selected from any one of the panels included in Table 4. In one embodiment, the panel is TNFR1, TNFR2, and IL-12p40/70. On other embodiments, once a diagnosis of CTCL is made, the method further comprises treating the subject for CTCL.

In one embodiment, the level of expression of one of more biomarkers of the invention in a sample is determined to be increased if the biomarker level is equal or above the upper normal limit, wherein the upper normal limit is defined as mean plus 2 standard deviations of a control.

In certain, non-limiting embodiments, the level of expression of one of more biomarkers of the invention in a sample is determined to be increased if the biomarker level is greater than about 1300 pg/ml for TNFR1, greater than about 1700 pg/ml for TNFR2, or greater than about 350 pg/ml for IL-12p70.

In one embodiment, an increase in the level of expression of one or more biomarkers of the invention or a nucleic acid coding for one or more biomarkers of the invention in a sample is an increase of 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more, as compared to the level of expression of one or more biomarkers of the invention or a nucleic acid coding for one or more biomarkers of the invention in a control sample.

In another embodiment, an increase in the level of expression of one or more biomarkers of the invention or a nucleic acid coding for one or more biomarkers of the invention in a sample is an increase of about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60% to about 65%, about 65% to about 70%, about 70% to about 75%, about 75% to about 80%, about 80% to about 85%, about 85% to about 90%, about 90% to about 100% or more, as compared to the level of expression of one or more biomarkers of the invention or a nucleic acid coding for one or more biomarkers of the invention in a control sample.

In another embodiment, an increase in the level of expression of one or more biomarkers of the invention or a nucleic acid coding for one or more biomarkers of the invention in a sample is an increase of about 5%, 10%, 15%, 20%, 25%, 30%, 35%, 40%, 45%, 50%, 55%, 60%, 65%, 70%, 75%, 80%, 85%, 90%, 100% or more, as compared to the level of expression of one or more biomarkers of the invention or a nucleic acid coding for one or more biomarkers of the invention in a control sample.

Ranges provided herein are understood to be shorthand for all of the values within the range. For example, a range of 10% or more is understood to include any number, combination of numbers, or sub-range from about 10% and above. Likewise, a range of 10% to 15% is understood to include any number, combination of numbers, or sub-range from about 10% to about 15%.

In one embodiment, the information provided by the methods described herein can be used by the physician in determining the most effective course of treatment. An indication of a diagnosis of a CTCL would be desirably considered in conjunction with clinical features of a subject's presentation to confirm a diagnosis, for example the appearance of the skin lesions, e.g., plaques or patches, and/or histopathology and/or the presence of atypical lymphocytes in the blood, lymph nodes or visceral organs. A positive result showing increased expression of one or more of the biomarkers of the invention may be preceded or followed by one or more further diagnostic measure, for example, tissue histopathologic analysis with immunophenotyping of malignant lymphocytes, molecular biology testing for clonality, In one embodiment, the diagnostic methods described in PCT Publication No. WO2014/124267 (the contents of which are hereby incorporated by reference) may be performed, which includes the use of biomarkers TOX, PLS3, KIR3DL2, GATA3 and RUNX3, where increased expression of TOX, PLS3, KIR3DL2, and/or GATA3 is associated with CTCL and decreased expression of RUNX3 is associated with CTCL, relative to a control.

In another embodiment, one or more therapeutic measure to treat and/or monitor CTCL, including the administration of a CTCL therapeutic agent, is carried out.

In another embodiment, the methods for detection of one or more biomarkers can be used to monitor the response in a subject to treatment. In one specific, non-limiting embodiment, the present invention further provides a method of treatment including measuring the presence of one or more biomarkers of the present invention in a subject at a first timepoint, administering a therapeutic agent for CTCL, re-measuring the one or more biomarkers at a second timepoint, comparing the results of the first and second measurements and optionally modifying the treatment regimen based on the comparison. In one embodiment, the first timepoint is prior to an administration of the therapeutic agent, and the second timepoint is after said administration of the therapeutic agent. In one embodiment, the first timepoint is prior to the administration of the therapeutic agent to the subject for the first time. In one embodiment, the dose (defined as the quantity of therapeutic agent administered at any one administration) is increased or decreased in response to the comparison. In another embodiment, the dosing interval (defined as the time between successive administrations) is increased or decreased in response to the comparison, including total discontinuation of treatment.

In particular non-limiting embodiments, a CTCL therapeutic agent may be corticosteroid, retinoid, imiquimod, radiation, methotrexate, UV light, romidepsin (e.g., Istodax®), photophoresis, bexarotene (e.g., Targetin) or a bexarotene analog, pralatrexate (e.g., Folotyn®), bortezomib (e.g., Velcade®), denileukin diftitox (e.g., Ontak®), vorinostat (e.g., Zolinza®), mechlorethamine gel (e.g., Valchlor™ or nitrogen mustard), alemtuzumab (e.g., Campath®), liposomal doxorubicin, gemcitabine (e.g., Gemzar®), everolimus (e.g., Afinitor®), lenalidomide (e.g., Revlimid®), brentuximab vedotin (Adcetris®), panobinostat, forodesine, AP0866 (a.k.a. Daporinad), mogamulizumab (W0761), or a combination thereof. In particular non-limiting embodiments, the therapeutic agent is a histone deacetylase inhibitor. See also Lindahl, 2013, Journal of the European Academy of Dermatology and Venereology, 27:2, 163; Lessin et al, 2013, JAMA Dermatol. 149(1):25-32; Weberschock et al., 2012, Cochrane Database Syst. Rev. September 12; 9:CD008946; and Kim et al., 2003, Arch. Dermatol. 139(7):857-866.

### Biomarker Detection

A biomarker used in the methods of the invention can be identified in a biological sample using any method known in the art. Determining the presence and/or level of one or more biomarker, e.g., protein or degradation product thereof, the presence and/or level of mRNA or pre-mRNA, or the presence and/or level of any biological molecule or product that is indicative of biomarker expression, or degradation product thereof, can be carried out for use in the methods of the invention by any method described herein or known in the art. In one embodiment, detection of the presence and/or level of one or more biomarker in the sample by a method described herein or known in the art transforms the sample.

Protein Detection Techniques

Methods for the detection and/or level of protein biomarkers are well known to those skilled in the art, and include but are not limited to bead-based multiplexing technology, e.g., xMAP® technology (Luninex Corporation), microarrays, (e.g., protein microarrays), mass spectrometry techniques, 1-D or 2-D gel-based analysis systems, chromatography, enzyme linked immunosorbant assays (ELISAs), radioimmunoassays (RIA), enzyme immunoassays (EIA), western blotting, immunoprecipitation, and immunohistochemistry. These methods use antibodies, or antibody equivalents, to detect protein. Antibody arrays, beads, or protein chips can also be employed, see for example U.S. Patent Application Nos. 20030013208A1; 20020155493A1, 20030017515 and U.S. Pat. Nos. 6,329,209 and 6,365,418, herein incorporated by reference in their entirety. ELISA and RIA procedures can be conducted such that a biomarker standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In one embodiment, a method for measuring biomarker expression includes the steps of: contacting a biological sample, e.g., blood, with a reagent, e.g., an antibody or variant (e.g., fragment) thereof, which selectively binds the biomarker, thereby transforming the sample in a manner such that the level of expression of the biomarker is detected and quantified, e.g., by detecting whether the reagent is bound to the sample. A method can further include contacting the sample with a second reagent, e.g., antibody, e.g., a labeled antibody. The method can further include one or more steps of washing, e.g., to remove one or more reagents.

It can be desirable to immobilize one component of the assay system on a support, such as a bead, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

The xMAP technology (Luminex Corp.), and similar multiplexed bead-based systems can also be used to measure the expression of the biomarkers of the invention. This technology combines the principle of a sandwich immunoassay with fluorescent bead-based technology, allowing individual and multiplex analysis of many different analytes, e.g., up to 100, in a single microtiter well (see Vignali D A. Multiplexed particle-based flow cytometric assays. J Immunol Methods 2000; 243:243-55 andYurkovetsky Z R, Kirkwood J M, Edington H D, et al. Clin Cancer Res. 2007; 13(8):2422-2428 for a detailed description).

Other techniques can be used to detect a biomarker according to a practitioner's preference based upon the present invention. One such technique is western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting and quantitating biomarker protein levels is western blotting. Cells can be frozen and homogenized in lysis buffer. Immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against biomarkers can also be used for imaging purposes, for example, to detect the presence of a biomarker in a sample of a subject. Suitable labels include radioisotopes, iodine (¹²⁵I, ¹²¹I), carbon (¹⁴C), sulphur (³⁵S), tritium (³H), indium (¹¹²In), and technetium (^{99m}Tc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In some embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., 13C) thereby permitting the test sample to be mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomarkers present in a sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to better quantify the amount of the biomarker present in the first sample. In certain embodiments, the identity of the biomarker in the first and second sample can also be determined.

RNA Detection Techniques

Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). In one embodiment, the sample being tested is transformed when the nucleic acid biomarker is detected, e.g., by Northern blotting or by amplification of the biomarker in the sample, in a manner such that the level of expression of the biomarker is detected and quantified.

In one embodiment, quantitative real-time polymerase chain reaction (qRT-PCR) is used to evaluate mRNA levels of biomarker. In one specific embodiment, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect mRNA levels of a biomarker by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. FISH test also can be automated.

Alternatively, mRNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA can be extracted from the biological sample to be tested, reverse transcribed and fluorescent-labeled cDNA probes are generated. The microarrays capable of hybridizing to a biomarker, cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, more preferably 18 bases and still more preferably 20 bases are preferred. Preferably, the primers and probes hybridize specifically under stringent conditions to a nucleic acid fragment having the nucleotide sequence corresponding to the target gene. As herein used, the term “stringent conditions” means hybridization will occur only if there is at least 95% and preferably at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

### Kits

In non-limiting embodiments, the present invention provides for a kit for determining whether a subject has CTCL comprising a means for detecting the biomarkers of the invention. The invention further provides for kits for determining the efficacy of a therapy for treating CTCL in a subject.

Types of kits include, but are not limited to, bead-based multiplexing technology, e.g., xMAP® technology (Luninex Corporation), packaged probe and primer sets (e.g. TaqMan probe/primer sets), arrays/microarrays, biomarker-specific antibodies and beads, which further contain one or more probes, primers or other detection reagents for detecting one or more biomarkers of the present invention.

In other non-limiting embodiments, a kit can comprise at least one antibody for immunodetection of the biomarker(s) to be identified. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In a further non-limiting embodiment, the biomarker-specific antibody can be provided bound to a solid support, such as a column matrix, an array, or well of a microtiter plate. Alternatively, the support can be provided as a separate element of the kit.

In a specific, non-limiting embodiment, a kit can comprise a pair of oligonucleotide primers suitable for polymerase chain reaction (PCR) or nucleic acid sequencing, for detecting one or more biomarker(s) to be identified. A pair of primers can comprise nucleotide sequences complementary to one or more biomarker of the invention. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also comprise one or more polymerases, reverse transcriptase and nucleotide bases, wherein the nucleotide bases can be further detectably labeled.

In non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In a further non-limiting embodiment, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise one or more reagents, e.g., primers, probes, microarrays, or antibodies suitable for detecting expression levels of markers selected from CXCL9, CXCL10, IL-12p40/70, CCL11, sIL-2R, CCL2, TNFR1, and TNFR2. In certain, non-limiting embodiments, a kit can comprise reagents for detecting expression levels of a panel of at least three of CXCL9, CXCL10, IL-12p40/70, CCL11, sIL-2R, CCL2, TNFR1, and NFR2. In another embodiment, a kit can comprise reagents for detecting expression levels of a panel comprising TNFR1, TNFR2, and IL-12p40/70.

In another non-limiting embodiment, a kit can comprise reagents for detecting expression levels of a panel of three or four biomarkers selected from the group consisting of TNFR1, TNFR2, IL12p40/70, CCL2, CCL11, and CXCL10, wherein the three or four biomarkers include one or both of TNFR1 and TNFR2, and wherein the remaining biomarkers include one or both of IL12p40/70 and CCL2 but not CXCL9, CXCL10, or sIL-2R.

In other non-limiting embodiments, a kit can comprise reagents for detecting expression levels of a panel of at least four of CXCL9, CXCL10, IL-12p40/70, CCL11, sIL-2R, CCL2, TNFR1, and TNFR2.

In another non-limiting embodiment, a kit can comprise reagents for detecting expression levels of a panel of three or four biomarkers selected from the panels of biomarkers identified in Table 4.

A kit can further contain means for comparing the biomarker with a control or reference, and can include instructions for using the kit to detect the biomarker of interest. Specifically, the instructions describes that the increase in the level of expression biomarker, e.g., as compared to a control sample, including a panel of biomarkers as set forth herein, is indicative that the subject has CTCL.

The following Examples are offered to more fully illustrate the invention, but are not to be construed as limiting the scope thereof.

### EXAMPLES

### Example 1

### Multimarker Assay for Early Detection of Mycosis Fungoides (MF)

This Example describes the identification of biomarkers that can be used for the differential diagnosis of early stage mycosis fungoides (MF).

### Materials and Methods

### Study Population

Blood from 30 patients with an established diagnosis of mycosis fungoides across all stages was collected. The blood was collected only from treatment naïve patients or patients with the active (progressive on the current treatment) disease. Eleven patients had an early stage disease (stage IA-IIA) and 19 patients had an advanced stage disease (stage IIB and above). Diagnosis of MF was established clinically and confirmed histologically according to criteria proposed by the International Society of Cutaneous Lymphoma. (Pimpinelli et al. J. Am. Acad Dermatol 2005; 53(6):1053-1056). Monoclonal T cell receptor gene rearrangement was detected in all patients by southern blot and PCR. A phenotype of malignant cells was determined by flow cytometry and revealed a diminished CD7 and CD26 expression on malignant lymphocytes in all patients.

726 healthy volunteers matched by sex and age served as the control group. Additionally, 10 patients with psoriasis were included as a validation group to control for the impact of cutaneous inflammation on biomarker expression.

### Luminex Assay Specifications and Procedure

Peripheral blood was collected from 30 patients with MF and processed within 30 minutes after the draw. 1 ml serum aliquots were prepared and stored at −80° C. prior to the Luminex analysis. A series of proteins (Table 2) were analyzed by xMap™ technology (Luminex Corporation) using kits from Invitrogen (Life Technologies, NY) according to the manufacturer's instructions as previously described (Yurkovetsky Z R, Kirkwood J M, Edington H D, et al. Clin Cancer Res. 2007; 13(8):2422-2428). Analysis of data was done using four-parametric-curve fitting (Little J A. Chromatographia 2004; 59:S177-S181).

TABLE 2
Biomarker Panel

| Groups | Markers |
|---|---|
| Apoptotic molecule | DR5 |
| Growth Factors | GM-CSF, EGF, FGFb, G-CSF, HGF, VEGF |
| Cytokines/ | IL-1B, IL-2, IL-4, IL-5, IL-6, IL-7, IL-8, |
| Cytokine receptors | IL-10, IL-12p40/70, IL-13, IL-15, IL-17, |
| | IFN-α, IFN-γ, TNF-α, TNFR1, TNFR2, |
| | sIL-2R, IL-1Ra |
| Chemokines/ | MCP-1, MIP-1a, MIP-1B, CCL11, RANTES, |
| Chemokine receptors | CXCL10, CXCL9, GROa |

### Immunohistochemistry

Histologically confirmed, formalin fixed paraffin embedded biopsies were collected from the inventors' institutional tissue bank. Human small bowel tissues were used as positive controls. Commercial antibodies were used for IL-12 staining (ab124635, dilution 1:500, Abcam Cambridge, Mass.). After de-paraffinization, heat mediated epitope retrieval was conducted for 60 minutes at 95° (Borg, Biocare Medical, Concord, Calif.). Endogen peroxidase activity was quenched with 3% hydrogen peroxide for 10 min. Prior blocking with Avidin/Biotin Blocking Kit (Vector Laboratories, Inc., Burlingame, Calif.), slides were incubated with normal rabbit serum for 20 minutes. Slides were incubated with primary antibodies for 30 minutes, the biotinylated polyclonal goat anti-rabbit secondary antibody (E0466, Dako, Carpinteria, Calif.) were incubated for 30 minutes, and then 4plus Streptavidin-AP Label (Ap605H; Biocare Medical, Concord, Calif.) was incubated for 30 minutes. Warp Red Chromagen was applied for 15 minutes (Biocare Medica, Concord, Calif.). Counterstaining was performed with Harris Hematoxylin for 15 seconds.

### Immunohistochemistry Score (IHC Score)

Immunostaining was scored using a previously established scoring system developed by Allred et al. Prognostic and predictive factors in breast cancer by immunohistochemical analysis. Modern pathology: an official journal of the United States and Canadian Academy of Pathology, Inc. 1998; 11(2):155-68. The percentage of stained cells was estimated on a scale of 0 to 100. Staining intensity was rated as negative (0), weak (1), intermediate (2), or strong (3). A total IHC score was calculated by adding the percentage multiple by the intensity score. The range of values is 0 to 300.

### Statistical Analyses

Markers with several 0's were dichotomized (0.1 vs. >1). For the markers that were dichotomized, McNemar's test was used to determine whether the proportions of marker levels that were 0.1 or >1 were the same in cases and controls. The markers that were not dichotomized were log-transformed so that they would be normally distributed. Paired t-tests were used to determine if the mean marker levels differed among cases and controls. Plots were created for marker levels that differed significantly among cases and controls (p<0.10).

Based on previously published data, the expected range of TNFR1 in controls is 770.2±411.2 pg/ml (Geskin et al. Exp Dermatol. 2014; 23(8): 598-600). A total of 30 patients entered this study. The probability was 90% that the study would detect a difference in a level of TNFR1 at a two-sided 0.05 significance level if the true difference between control group and patients was 505.1 pg/ml. This was based on the assumption that the standard deviation of the TNFR1 was 411.2 pg/ml.

A Metropolis algorithm (MA) was used for analysis of the data (Yurkovetsky Z, Ta'asan S, Skates S, et al. Gynecol Oncol. 2007; 107(1):58-65). In MA analysis, the scoring function for a particular biomarker panel was constructed as a linear combination of number of positive test for each biomarker. The biomarker was considered to be positive if the biomarker level was equal or above the upper normal limit that was defined as mean plus 2 standard deviations in the control group of healthy volunteers. Only the panels where all tested biomarkers were positive were used for calculation of sensitivity (SN) and specificity (SP). The cutoff was adjusted at the each iteration of parameter estimates to maintain the desired SP (>95%). No patient cases were excluded from this analysis. All possible panels consisting of two, three, and four biomarkers were evaluated for SN at 90% SP in the preliminary training set.

Overall survival (OS) was defined as the time from first day of diagnosis to death from any cause. Patients without an event in OS were censored at the last day with valid information for the respective endpoint. OS were estimated according to Kaplan-Meier and compared by log-rank (Mantle-Cox) trend test.

Multivariate analyses were conducted with the use of Cox proportional hazard models to estimate hazard ratios (HRs) for evolving an event. The nominal significance level was at 0.05 two-sided. The inventors are aware of the problem of multiple comparisons and, therefore, have chosen to extract the most prominent aspect.

### Results

### Clinical Characteristics of the Patient Population

In total, 30 patients (15 male, 15 female; median age 65 years) with established diagnosis of MF were included in this study. The clinical characteristics of 30 patients are listed in Table 3. Eleven patients had an early stage disease (stage IA-IIA), and 19 patients had an advanced stage disease (stage IIB and above). 7 out of 11 patients with early MF had patch stage disease. None of the patients with early MF had blood involvement; while 8 out of 19 patients with advanced disease had circulating malignant lymphocytes at the leukemic level.

TABLE 3
Patient characteristics

All patients
(N = 30)

| Characteristics | No. | % |
|---|---|---|
| Age, years | | |

| | |
|---|---|
| Median | 64.5 |
| Range | 31-87 |

| | | |
|---|---|---|
| Sex | | |
| Male | 15 | 50.0 |
| Female | 15 | 50.0 |
| MF, early | 11 | 36.7 |
| Patches (T1a) | 7 | 63.6 |
| Plaques (T1b) | 4 | 36.4 |
| MF, advanced | 19 | 63.3 |
| Tumors (T3) | 5 | 26.3 |
| Leukemia (B2) | 8 | 42.1 |

### Biomarker Selection for Multimarker Panel

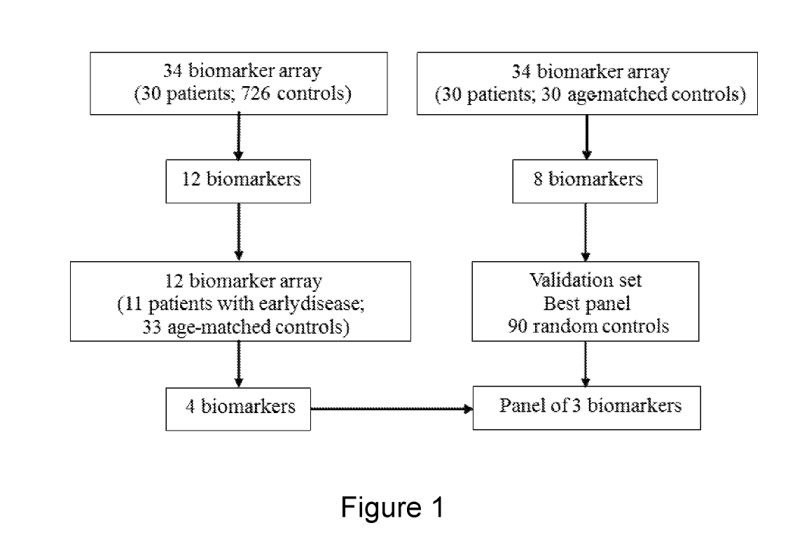

Because of the small prevalence of MF, the specificity of serum tests should be high (>95%). Accurate assessment of such high specificity requires large number of healthy participant/normal samples; thus, a dataset of 726 healthy volunteers was utilized. First, a broad preliminary screening to select a subset of biomarker combinations (panels) was performed (FIG. 1).

Analysis of biomarkers in 30 MF patients vs. 726 healthy volunteers revealed 12 biomarkers, which had a level of expression which was significantly different (p<0.05) between those two groups. The following 12 biomarkers were identified: CXCL9, CXCL10, IL-12p40/70, MIP1B, CCL11, sIL-2R, CCL2, RANTES, FGFB, HGF, TNFR1, and TNFR2.

The inventors have previously demonstrated that distinguishing between normal immunosenescence and cancer-related changes is important when evaluating cytokine profile in cancer patients (Geskin L J, Akilov O E, Lin Y, Lokshin A E. Exp Dermatol. 2014; 23(8): 598-600). To eliminate age-related changes in the immune system, 30 controls of the same age as our patient group were randomly selected out of 726 healthy volunteers available for this study (age-matched controls). The expressions were significantly different among cases and controls for the following eight biomarkers: CXCL9, CXCL10, IL-12p40/70, CCL11, sIL-2R, CCL2, TNFR1, TNFR2 (p<0.05). The difference in four biomarkers (MIP1B, RANTES, FGFB, and HGF) was attributed to age-dependent immunosenescence rather than being disease-specific and therefore were not included in overall analysis.

### Statistical Analysis of Multimarker Panel

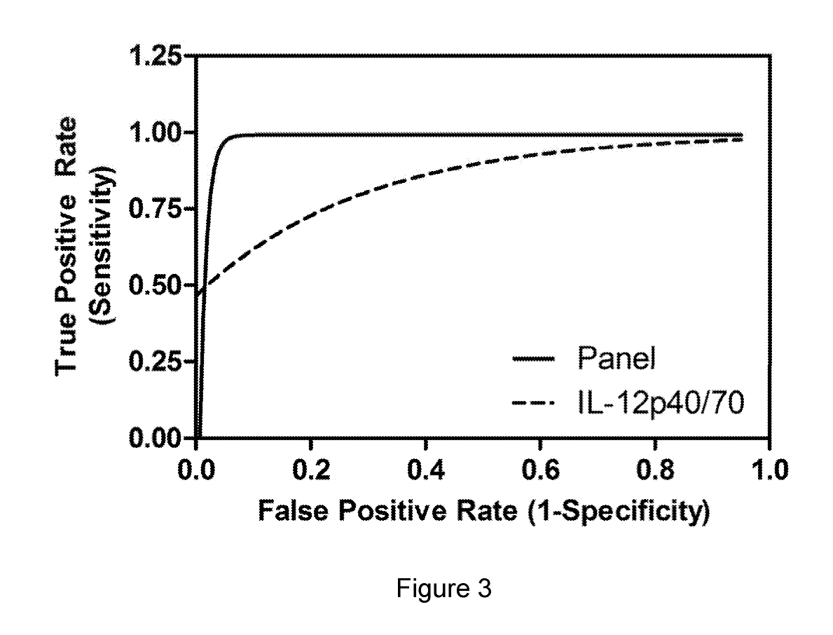

The performance of two-, three-, and four-biomarker panels was compared. Three-biomarker panels offered superior performance compared with two-biomarkers panels, whereas using four-biomarker panels did not result in significant improvement. Six four-biomarker panels with highest specificity (>95%) for MF were identified. These panels represented various combinations of the following biomarkers: TNFR1, TNFR2, sIL-2R, CCL2, CXCL9, CXCL10, and IL-12p40/70 (Table 4). Three three-biomarker panels were identified with specificity >95% (TNFR1, TNFR2, and IL-12p40/70; TNFR1, TNFR2, and sIL-2R; and TNFR1, TNFR2, and CXCL9). Among them, the combination of TNFR1, TNFR2, and IL-12p40/70 provided sensitivity of 86.7% for overall MF and 72.7% for early MF disease. FIG. 3 demonstrates cumulative receiver operating characteristic (ROC) curves using the three-biomarker panel (TNFR1, TNFR2, and IL-12) vs. IL-12p40/70 in patients with MF vs. healthy controls. For comparison, the sensitivity of IL-12p40/70 alone at 72.2% specificity was 90.9% for early-stage disease and 93.3% for MF without differentiation for stages.

TABLE 4
Sensitivities of Different Panels of Biomarkers After Cross Validation

| | | | | | | Early MF |
|---|---|---|---|---|---|---|
| Biomarker Panels | | | | All MF (n = 30) | | (n = 11) |
| M1 | M2 | M3 | M4 | Specificity | Sensitivity | Sensitivity |
| TNFR1 | TNFR2 | IL-12p40/70 | | 97.8 | 86.7 | 72.7 |
| TNFR1 | TNFR2 | CCL2 | | 91.1 | 83.3 | 63.6 |
| CCL2 | TNFR2 | IL-12p40/70 | | 90.0 | 83.3 | 72.7 |
| TNFR1 | CCL2 | IL-12p40/70 | | 88.9 | 83.3 | 72.7 |
| TNFR1 | TNFR2 | IL-12p40/70 | CCL2 | 93.3 | 80.0 | 63.6 |
| TNFR1 | CCL2 | IL-12p40/70 | CCL11 | 90.0 | 80.0 | 63.6 |
| TNFR1 | TNFR2 | IL-12p40/70 | CCL11 | 94.4 | 76.7 | 63.6 |
| TNFR1 | TNFR2 | CXCL10 | | 93.3 | 76.7 | 63.6 |
| TNFR1 | TNFR2 | CCL11 | | 88.9 | 76.6 | 63.6 |
| TNFR1 | TNFR2 | CCL2 | CXCL10 | 93.3 | 70.0 | 54.5 |
| TNFR1 | TNFR2 | CCL2 | CCL11 | 92.2 | 70.0 | 54.5 |
| TNFR1 | CCL2 | IL-12p40/70 | CXCL10 | 95.6 | 66.7 | 54.5 |
| TNFR1 | TNFR2 | sIL-2R | | 97.8 | 53.3 | 18.2 |
| TNFR1 | TNFR2 | IL-12p40/70 | sIL-2R | 98.9 | 50.0 | 18.2 |
| TNFR1 | TNFR2 | IL-12p40/70 | CXCL10 | 95.6 | 50.0 | 18.2 |
| sIL-2R | TNFR2 | CCL2 | IL-12p40/70 | 100.0 | 46.7 | 18.2 |
| TNFR1 | CCL2 | IL-12p40/70 | sIL-2R | 98.9 | 46.7 | 18.2 |
| TNFR1 | TNFR2 | CXCL9 | | 95.6 | 46.7 | 45.5 |
| TNFR1 | CCL2 | IL-12p40/70 | CXCL9 | 97.8 | 43.3 | 45.5 |

Association of Circulating TNFR1, TNFR2, and IL-12p40/70 Levels with Survival of MF Patients

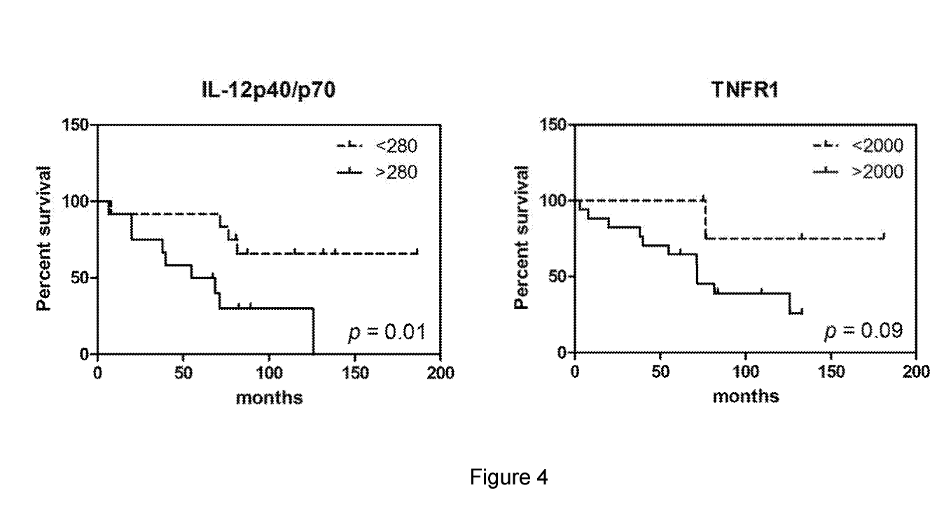

Biomarkers that are tightly linked to the pathogenesis of MF reflect, to some extent, the aggressive behavior of MF and thereby influence the overall specific survival of patients with MF. Therefore, overall specific survival of patients with MF was compared with high and low serum levels in univariate analyses. A strong association of circulating level of IL-12p40/70 with OS was observed. Patients with MF with serum level of IL-12p40/70 >280 pg/ml had significantly shorter overall survival (OS) (p=0.0102) (see FIG. 4). While median survival of patients with MF, who had >2000 pg/ml of circulating TNFR1 was 71.5 months and median survival in the group of <2000 pg/ml of TNFR1 was not identified, statistical analysis failed to demonstrate a significant difference between the groups of high and low TNFR1 levels (p=0.0968).

Multivariate analysis adjusted for the clinical characteristics was conducted to identify biomarkers with prognostic power independent of the clinical features. In Table 5, the results of this multivariate analysis are shown for TNFR1, TNFR2, and IL-12p40/70. An increased HR of TNFR1 for shorter OS was observed in elderly patients and patients with aggressive disease (p<0.05 in both groups). The respective Cox model for IL-12p40/70 revealed the HR for shorter OS was comparable with the HR of being elderly and have an aggressive disease (p<0.05 in both groups). For the TNFR2, no association with OS of patients with MF were observed in multivariate analysis.

TABLE 5
Multivariate analysis of TNFR1, TNFR2, and IL-12
in relation to OS adjusted to the patients'
characteristics and clinical factors (Cox model)

| Factor | HR (95% CI) | p |
|---|---|---|
| logTNFR1 | | |
| Sex | 9.02e−01 (9.14e−05 − 8.89e+03) | 0.986 |
| Race | 5.83e−01 (6.57e−05 − 5.18e+03) | 0.908 |
| Stage at Dx | 2.52e−01 (1.31e−02 − 4.85e+00) | 0.362 |
| Age at Dx | 2.27e+00 (1.07e+00 − 4.80e+00) | 0.032 |
| Stage at blood draw | 1.08e+02 (4.49e−01 − 2.63e+04) | 0.094 |
| Aggressive course | 7.20e+06 (1.14e+00 − 4.54e+13) | 0.048 |
| Patch/plaques or tumors | 1.95e+04 (8.57e−01 − 4.42e+08) | 0.054 |
| BSA | 8.67e−01 (7.14e−01 − 1.05e+00) | 0.150 |
| mSWAT | 9.22e−01 (8.15e−01 − 1.04e+00) | 0.196 |
| logTNFR2 | | |
| Sex | 4.34e+02 (6.71e−03 − 2.81e+07) | 0.283 |
| Race | 9.73e+00 (0.13e−04 − 8.37e+05) | 0.695 |
| Stage at Dx | 2.93e−01 (1.39e−02 − 6.18e+00) | 0.430 |
| Age at Dx | 1.93e+00 (8.02e−01 − 4.65e+00) | 0.142 |
| Stage at blood draw | 3.41e+01 (5.64e−02 − 2.07e+04) | 0.280 |
| Aggressive course | 2.15e+12 (1.21e−02 − 3.81e+26) | 0.090 |
| Patch/plaques or tumors | 2.39e+01 (1.92e−05 − 2.98e+07) | 0.658 |
| BSA | 1.59e+00 (8.98e−01 − 2.80e+00) | 0.113 |
| mSWAT | 8.61e−01 (6.44e−01 − 1.15e+00) | 0.313 |
| logIL-12p40/70 | | |
| Sex | 1.78e+01 (1.20e−03 − 2.66e+05) | 0.557 |
| Race | 9.11e−03 (3.24e−06 − 2.56e+01) | 0.246 |
| Stage at Dx | 1.44e+00 (2.02e−01 − 1.03e+01) | 0.717 |
| Age at Dx | 1.69e+00 (1.06e+00 − 2.70e+00) | 0.029 |
| Stage at test | 2.37e+01 (4.52e−01 − 1.24e+03) | 0.117 |
| Aggressive course | 1.30e+04 (2.73e+00 − 6.23e+07) | 0.028 |
| Patch/plaques or tumors | 2.78e+02 (1.94e−01 − 4.0e+05) | 0.129 |
| BSA | 8.66e−01 (7.25e−01 − 1.04e+00) | 0.114 |
| mSWAT | 9.59e−01 (8.67e−01 − 1.06e+00) | 0.415 |

### Selectivity of the Panel for Mycosis Fungoides Versus Psoriasis

All three biomarkers (TNFR1, TNFR2, and IL-12p40/70), were found in higher concentrations in the patients with MF when compared to control group or to patients with psoriasis (FIG. 2A). While the level of TNFR1 in patients with psoriasis was higher than in the control group (1147.0±307.7 pg/ml vs. 770.2±411.2 pg/ml, p<0.05), the level of TNFR1 in patients with MF exceeded the level of TNFR1 in patients with psoriasis by 2.3 times (2670.0±944.3 pg/ml vs. 1147.0±307.7 pg/ml, p<0.001) and controls by 3.5 times (p<0.001).

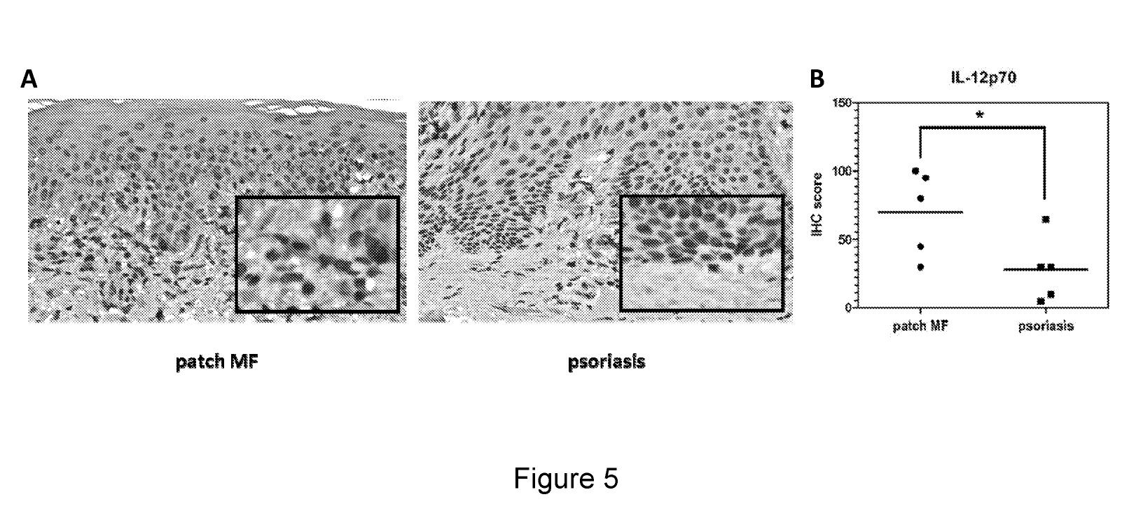

While the level of those proteins were increased with age, the difference between MF patients and controls were uniformly present across all age groups (see FIG. 2B). Immunohistochemistry of IL-12p70 in patches of MF and psoriasis vulgaris demonstrated distinguishing pattern equivalent to what was observed with circulating IL-12p70 level in those patients (see FIG. 5). The extracellular staining for IL-12p70 was significantly more intense in patients with MF in comparison with patients with psoriatic lesions.

Loss of TNFR1 and TNFR2 on Malignant Lymphocytes in Patients with Mycosis Fungoides and Sézary Syndrome

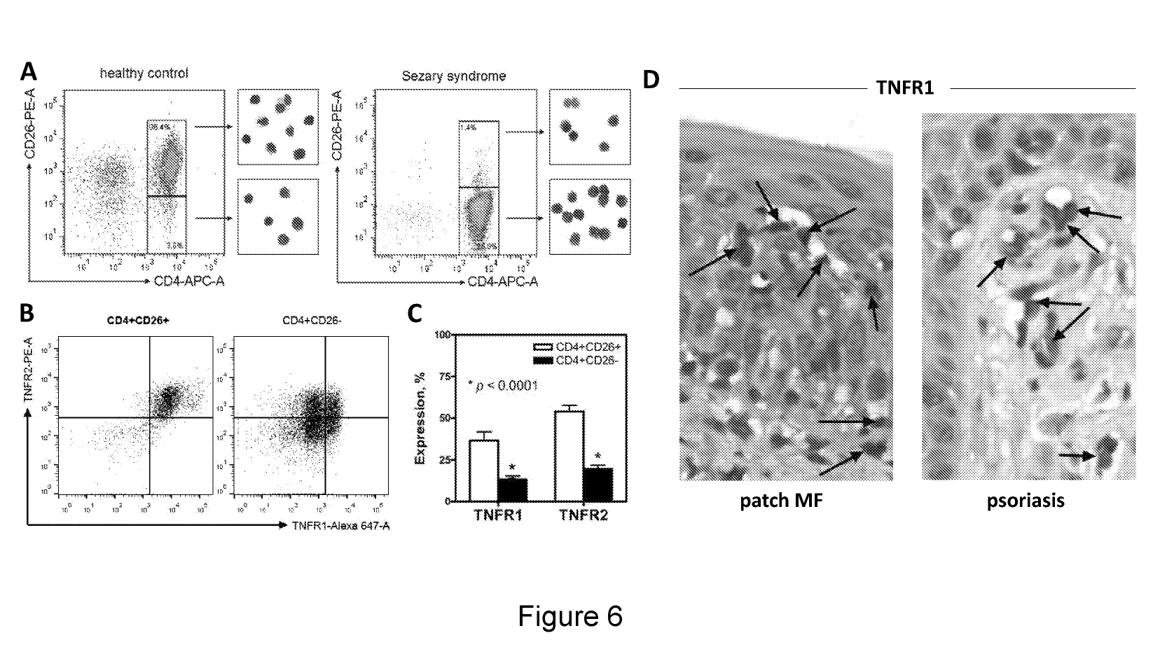

Malignant lymphocytes were isolated from the peripheral blood of patients with Sezary syndrome and separated from non-malignant cells based on the loss of CD26 expression. Morphology of these CD4⁺CD26⁻ cells derived from patients with Sezary cells revealed convoluted cerebriform nuclei characteristic of malignant cells (FIG. 6A) and in correspondence with previously published data (Bernengo M G et al. Br. J. Dermatol. 2001; 144(1):125-35). A significantly lower percentage of CD4⁺CD26⁻ cells expressed TNFR1 and TNFR2 when compared to CD4⁺CD26⁺ lymphocytes in the patient with Sezary syndrome (see FIGS. 6B and 6C). Interestingly, those findings were observed in the skin samples from patients with patch MF. Malignant lymphocytes forming Pautrie microabscess in the middle portion of the epidermis lacked TNFR1 in comparison with strong surface expression of TNFR1 on lymphocytes in patients with psoriasis (see FIG. 6D). Thus, the high level of soluble TNFR1 and TNFR2 in the serum of the patients with MF and Sezary syndrome together with the profound loss of TNFR1 and TNFR2 on the malignant lymphocytes signifies the shedding of these receptors from the malignant cells.

### Discussion

Biomarker discovery is a rapidly developing area of modern medicine. A particularly important application for this methodology is early detection of cancer. The goal of the present study described in this Example, was to select a panel of biomarkers that would provide high-level sensitivity and specificity for distinguishing early-stage MF from non-MF controls from a large array of serum proteins. Since MF is a malignancy of lymphocytes, the panel contained a broad range of lymphocyte-driven cytokines and chemokines as well as growth factors and apoptotic molecules, e.g., DR5, using a bead-based xMAP multiplexing technology which allows for detection of up to 100 biomarkers in 50 μl of a biologic sample.

The inventors have demonstrated previously that age-related immunosenescence needs to be taken into consideration when evaluating immune dysregulation in the elderly and should be performed with appropriate age matched controls (Geskin L J, Akilov O E, Lin Y, Lokshin A E. Exp Dermatol. 2014; 23(8); 598-600; Geskin et al. Blood 2015; 124(18):2798-805). Thus, the present study was conducted in an age-matched manner, taking into consideration age-related immunosenescence.

After performing xMAP multiplexing bead-based immunoassay screening, consistently elevated serum levels were observed for biomarkers CXCL9, CXCL10, IL-12p40/70, CCL11, sIL-2R, CCL2, TNFR1, and TNFR2. Consistent with previous observations of the high level of soluble TNFR1 in the serum of the patients with Sézary syndrome, together with the profound loss of TNFR1 on the malignant lymphocytes (Akilov O E, Wu M X, Ustyugova I V, Falo L D, Jr., Geskin L J. Exp Dermatol. 2012; 21(4):287-292), an elevation of TNFR1 in patients with MF was found as well. In addition, the patients with MF showed elevation of TNFR2. The release of the extracellular domain of TNFRs and the resulting decrease of the number of receptor molecules on the surface was shown to desensitize the cell for the TNF-α effects (Aderka D. Cytokine Growth Factor Rev. 1996; 7(3):231-240) and contribute to resistance of the malignant cells to apoptosis.

Using a Metropolis algorithm to analyze the data (Yurkovetsky Z, et al. Gynecol Oncol 2007; 107(1): 58-65), the three-biomarker panel providing the highest diagnostic power of 86% sensitivity (SN) for all stages of MF and 72% SN for early-stage of MF at 98% specificity (SP) was found to be comprised of TNFR1, TNFR2, and IL12p40/70. This dataset was validated using serum from the patients with benign skin dermatoses, such as psoriasis and demonstrated that this panel is specific for MF and not a non-specific marker of skin inflammation.

Accordingly, this biomarker panel has sufficient specificity and sensitivity for screening for early MF. This panel also allows for selection of a group of patients among many with non-specific dermatological manifestations for further assessment with current standard tests, including skin biopsies and molecular testing. Detection at an early stage of disease allows for appropriate treatment early, influencing the course of the disease significantly and providing measurable improvement in the quality of life of patients with MF.

### Example 2

### Multiplex Assay for CTCL

This Example describes the development and use of a multiplex Luminex® bead-based assay (MLBA) for determination of levels of a panel of three biomarkers, TNFR1, TNFR2 and IL-12p40/70 in human plasma or serum to diagnose CTCL. The multiplex assay performance can be compared with the corresponding single ELISA assay.

Comparison of the Performance of a Multiplex Bead-Based Assay for Determination of Three Biomarkers, TNFR1, TNFR2 and IL-12p40170 in Human Plasma or Serum with the Corresponding Singleplex ELISAs

A three-biomarker panel including TNFR1, TNFR2 and IL-12p40/70 is developed using reagents from the commercially available Luminex® bead-based panels. Prior to clinical validation, analytical validation of multiplexed immunoassay performance is conducted. Using standard and clinical samples, the following specific performance characteristics of the multiplex ELISA are defined and compared: range of linearity, analytical specificity, recovery, limits of detection and quantification (sensitivity), reasonable imprecision (precision), and comparison to a quality reference method.

Singleplex/conventional ELISA is a known method for highly sensitive qualitative and quantitative detection of analytes within heterogeneous samples. Therefore, the performance of a multiplex assays is compared with that of singleplex ELISAs to determine the corresponding analytes.

Commercially available singleplex ELISA kits are used for detection of TNFR1, TNFR2 and IL-12p40/70 (R&D Systems (Minneapolis, Minn.)). Calibration curves are prepared for multiplex and singleplex ELISAs consisting of a series of dilutions of the quality control (QC) standards with known protein concentrations that are plotted against assay signal, and a mathematical expression is fitted to the curve. Issues relevant to multiplexed protein immunoassay development include elimination of assay interference between reagents and configuration of assay sensitivities to provide acceptable dynamic ranges for each of the multiplexed proteins in the targeted specimen matrix (Kingsmore, S. F. Multiplexed protein measurement: technologies and applications of protein and antibody arrays. Nat Rev Drug Discov 5, 310-320 (2006)). Therefore, in order to reach maximum agreement with singleplex ELISA, the conditions for performing of the multiplex assay are optimized. Optimization of the assay parameters such as sample dilution factor, incubation times and washing steps are carried out.

Cross-reactivity between detection antibodies and immobilized capture antibodies and nonspecific analytes may also diminish the performance of the multiplex test. For example, some protein combinations may cause a significant increase in nonspecific binding, producing a large background signal, and thereby decrease assay sensitivity. Antibody-related assay interference is evaluated in each assay format with three experiments that measure the signal produced when (1) single proteins are incubated with complete detection antibody cocktail; (2) complete protein mixture is incubated with single detection antibodies; and (3) antibody cocktails with one antibody removed are incubated with complete protein mixtures to detect cross-reactivity between detection antibodies and specific proteins (Gonzalez, R. M. et al. Development and validation of sandwich ELISA microarrays with minimal assay interference. (J Proteome Res 7, 2406-2414 (2008)).

Accuracy and precision of the multiplex ELISA is estimated as follows. Accuracy: recovery studies are performed by mixing an aliquot of serum or plasma samples and analyte standards. The results obtained with multiplex ELISA (observed values) are compared with expected values. The percentage of recovery (accuracy) are calculated from the ratio of observed values to expected values. Precision: Inter-assay and Intra-assay coefficient of variation (CV) are evaluated at 20 serum and plasma samples for each antigen, using multiplex ELISA. Accuracy and precision of the multiplex assay is considered satisfactory if the percentage of recovery and inter-assay and intra-assay imprecision is <20%.

Optimization of the listed multiple assay variables are carried out using multifactorial design of experiments.

### Demonstration of the Use of the Multiplex Assay for Early Detection of CTCL in Human Serum or Plasma Using Clinical Samples

The objective of this study is to perform validation studies using a multiplex assay for determination of TNFR1, TNFR2 and IL-12p40/70 in human plasma or serum, as optimized above, for early diagnosis of CTCL.

Preliminary validation of the multiplex method comprises 1) statistical analysis (see below), and 2) interference studies. All statistical analyses are performed using SPSS software (Release 12, Chicago, Ill., USA). Clinical samples are obtained from CTCL patients and patients with benign dermatoses (eczema and psoriasis).

The measurement of the TNFR1, TNFR2 and IL-12p40/70 concentrations is performed using the optimized multiplex assay (described above) and data interpretation is performed as described above. In more detail, based on the previously published data, the probability that the study would detect a difference in a level of TNFR1, TNFR2 and IL-12p40/70 at a two-sided 0.05 significance level is calculated. These calculations are based on the previously calculated standard deviations for these markers.

A Metropolis algorithm (MA) is used for analysis of the data. (Gelfand, J. M. et al. J Invest Dermatol 126, 2194-2201 (2006); Gelfand, J. M., et al. Arch Dermatol 139, 1425-1429 (2003); and Zhang, Y. et al. Prior medical conditions and medication use and risk of non-Hodgkin lymphoma in Connecticut United States women. Cancer causes & control: CCC 15, 419-428 (2004)). In MA analysis, the scoring function for a specific biomarker panel is constructed as a linear combination of number of positive test for each biomarker. The biomarker is considered to be positive if the biomarker level will be equal or above the upper normal limit (defined as mean plus 2 standard deviations in the control group of healthy volunteers). Sensitivity (SN) and specificity (SP) is calculated for positive biomarkers. The cutoff is adjusted at each iteration of parameter estimates to maintain the desired SP (>95%). The comparison studies are performed in blinded manner.

This study demonstrates the multiplex system's ability to detect CTCL with clinical sensitivity, for example, of not less than about 85% and specificity, for example, of not less than about 95%.

### REFERENCES

1. Korgavkar K, Xiong M, Weinstock M. Changing incidence trends of cutaneous T-cell lymphoma. JAMA Dermatol. 2013; 149(11):1295-1299.
2. Arai E, Katayama I, Ishihara K. Mycosis fungoides and Sezary syndrome in Japan. Clinicopathologic study of 107 autopsy cases. Pathol Res Pract. 1991; 187(4):451-457.
3. Kim Y H, Hoppe R T. Mycosis fungoides and the Sezary syndrome. Semin Oncol. 1999; 26(3):276-289.
4. Foss F M, Sausville E A. Prognosis and staging of cutaneous T-cell lymphoma. Hematol Oncol Clin North Am. 1995; 9(5):1011-1019.
5. Duvic M, Apisarnthanarax N, Cohen D S, Smith T L, Ha C S, Kurzrock R. Analysis of long-term outcomes of combined modality therapy for cutaneous T-cell lymphoma. J Am Acad Dermatol. 2003; 49(1):35-49.
6. Santucci M, Biggeri A, Feller A C, Burg G. Accuracy, concordance, and reproducibility of histologic diagnosis in cutaneous T-cell lymphoma: an EORTC Cutaneous Lymphoma Project Group Study. European Organization for Research and Treatment of Cancer. Arch Dermatol. 2000; 136(4):497-502.
7. Zemheri E, Ozkanli S, Zindanci I, et al. Evaluation of histopathological changes in control biopsies which taken 48 sessions after NBUVB phototherapy for early-stage mycosis fungoides. Scientific World Journal. 2012; 2012:426732.
8. Lefeber W P, Robinson J K, Clendenning W E, Dunn J L, Colton T. Attempts to enhance light microscopic diagnosis of cutaneous T-cell lymphoma (mycosis fungoides). Arch Dermatol. 1981; 117(7):408-411.
9. Olerud J E, Kulin P A, Chew D E, et al. Cutaneous T-cell lymphoma. Evaluation of pretreatment skin biopsy specimens by a panel of pathologists. Arch Dermatol. 1992; 128(4):501-507.
10. Tok J, Szabolcs M J, Silvers D N, Zhong J, Matsushima A Y. Detection of clonal T-cell receptor gamma chain gene rearrangements by polymerase chain reaction and denaturing gradient gel electrophoresis (PCR/DGGE) in archival specimens from patients with early cutaneous T-cell lymphoma: correlation of histologic findings with PCR/DGGE. J Am Acad Dermatol. 1998; 38(3):453-460.
11. Geskin L J, Akilov O E, Lin Y, Lokshin A E. Distinct Age-Matched Serum Biomarkers Profiles in Patients with Cutaneous T-Cell Lymphoma. Exp Dermatol. 2014; 23(8): 598-600.
12. Vignali D A. Multiplexed particle-based flow cytometric assays. J Immunol Methods. 2000; 243(1-2):243-255.
13. Yurkovetsky Z R, Kirkwood J M, Edington H D, et al. Multiplex analysis of serum cytokines in melanoma patients treated with interferon-alpha2b. Clin Cancer Res. 2007; 13(8):2422-2428.
14. Little J A. Comparison of curve fitting models for ligand binding assays. Chromatographia 2004; 59:S177-S181.
15. Yurkovetsky Z, Ta'asan S, Skates S, et al. Development of multimarker panel for early detection of endometrial cancer. High diagnostic power of prolactin. Gynecol Oncol. 2007; 107(1):58-65.
16. Akilov O E, Wu M X, Ustyugova I V, Falo L D, Jr., Geskin L J. Resistance of Sezary cells to TNF-alpha-induced apoptosis is mediated in part by a loss of TNFR1 and a high level of the IER3 expression. Exp Dermatol. 2012; 21(4):287-292.
17. Aderka D. The potential biological and clinical significance of the soluble tumor necrosis factor receptors. Cytokine Growth Factor Rev. 1996; 7(3):231-240.
18. Zackheim, et al. J. Am. Acad Dermatol 2002; 47(6):914-8.
19. Pimpinelli et al. J. Am. Acad Dermatol 2005; 53(6):1053-1056.
20. Allred et al. Prognostic and predictive factors in breast cancer by immunohistochemical analysis. Modern pathology: an official journal of the United States and Canadian Academy of Pathology, Inc. 1998; 11(2):155-68.
21. Bernengo M G et al. Br. J. Dermatol. 2001; 144(1):125-35.
22. Yurkovetsky Z, et al. Gynecol Oncol 2007; 107(1): 58-65.
23. Olek-Hrab K, Silny W. Rep Pract Oncol Radiother 2014; 19(2): 72-6.
24. Geskin et al. Blood 2015; 124(18):2798-805.
25. Ungewickell A, Bhaduri A, Rios E, et al. Nat Genet 2015; 47(9): 1056-60.

The contents of Uniprot Accession Numbers, Figures, and various publications, patents and patent applications which are cited herein are hereby incorporated by reference herein in their entireties.

What is claimed is:

1. A method of determining whether a subject has cutaneous T-cell lymphoma (CTCL), the method comprising determining the levels of three or more biomarkers in a biological sample obtained from the subject relative to the level of expression of the three or more biomarkers in a control sample, wherein the biomarkers comprise TNFR1, TNFR2, and IL12p40/70, and wherein increased expression of the three or more biomarkers indicates a diagnosis of cutaneous T-cell lymphoma in the subject.

2. The method of claim 1, further comprising the step of performing an additional CTCL detection method.

3. A method of excluding a diagnosis of cutaneous T-cell lymphoma (CTCL), or an associated disorder, in a subject, comprising determining the levels of three or more biomarkers in a biological sample obtained from the subject relative to the level of expression of the three or more biomarkers in a control sample, wherein the biomarkers comprise TNFR1, TNFR2, and IL12p40/70, and wherein normal levels of the three or more biomarkers excludes a diagnosis of CTCL.

4. The method of claim 3, wherein the subject is treated with a therapeutic agent that is useful for the treatment of a skin disorder other than CTCL.

5. The method of claim 1 or 2, comprising obtaining a biological sample from the subject.

6. The method of claim 1 or 2, wherein the biological sample is a blood sample.

7. The method of claim 6, wherein the blood sample is a serum sample or a plasma sample.

8. The method of claim 1 or 2, wherein the subject is a human.

9. The method of claim 1 or 2, wherein the subject has a skin lesion.

10. The method of claim 1 or 2, wherein the cutaneous T-cell lymphoma (CTCL) is mycosis fungoides (MF) or Sézary syndrome (SS).

11. The method of claim 10, wherein the MF or SS is early stage MF or SS.

12. The method of claim 1 or 2, wherein the three or more biomarkers further comprise at least one of CXCL9, CXCL10, CCL11, sIL-2R, and CCL2.

13. The method of claim 1 or 2, wherein TNFR1, TNFR2, and IL12p40/70 are proteins.

14. The method of claim 13, wherein the level of the proteins in the biological sample is detected by contacting the sample with a reagent which specifically binds with the proteins.

15. The method of claim 14, wherein the reagent is an antibody or antigen-binding fragment thereof.

16. The method of claim 15, wherein the antibody or antigen-binding fragment thereof is a monoclonal antibody.

17. The method of claim 1, wherein the method further comprises treating the subject with a therapeutic agent for CTCL.

18. A method of assessing the efficacy of a therapy for treating cutaneous T-cell lymphoma (CTCL) in a subject, comprising:
(a) determining the levels of three or more biomarkers in a biological sample obtained from the subject, prior to therapy with a therapeutic agent, wherein the biomarkers comprise TNFR1, TNFR2, and IL12p40/70; and
(b) determining the levels of the three or more biomarkers in a biological sample obtained from the subject, at one or more time points during therapy with the therapeutic agent,
wherein the therapy with the therapeutic agent is efficacious for treating the cutaneous T-cell lymphoma in the subject when there is a lower level of the three or more biomarkers in the second or subsequent samples, relative to the first sample.

19. The method of claim 18, wherein the therapeutic agent is selected from the group consisting of: corticosteroid, retinoid, imiquimod, radiation, methotrexate, UV light, romidepsin, photophoresis, bexarotene or a bexarotene analog, pralatrexate, bortezomib, denileukin diftitox, vorinostat, mechlorethamine gel, nitrogen mustard, alemtuzumab, liposomal doxorubicin, gemcitabine, everolimus, lenalidomide, brentuximab vedotin, panobinostat, forodesine, AP0866, mogamulizumab (W0761), a histone deacetylase inhibitor, or a combination thereof.

20. A kit for diagnosing whether a subject has cutaneous T-cell lymphoma (CTCL) or assessing the efficacy of a therapeutic agent for the treatment of CTCL, comprising reagents useful for detecting three or more biomarkers, wherein the biomarkers comprise TNFR1, TNFR2, and IL12p40/70 in a biological sample from the subject.

21. The kit of claim 20, comprising one or more of packaged arrays/microarrays, biomarker-specific antibodies, or beads.

22. The kit of claim 21, comprising at least one monoclonal antibody or antigen-binding fragment thereof, for detecting the biomarkers to be identified.

23. The kit of claim 20, wherein the cutaneous T-cell lymphoma (CTCL) is mycosis fungoides (MF) or Sézary syndrome (SS).

24. The kit of claim 20, wherein the three or more biomarkers further comprises at least one of CXCL9, CXCL10, CCL11, sIL-2R, and CCL2.
